(19) United States
(12) Patent Application Publication
(10) Pub. No.: US 2011/0280815 A1
(43) Pub. Date: Nov. 17, 2011

(54) COMPOSITION FOR INHIBITING MELANIN PRODUCTION AND APPLICATION THEREOF

(75) Inventors: Han-Fen HUANG, Kaohsiung (TW); Shan-Ying Lin, Kaohsiung (TW)

(73) Assignee: TAIWAN HOPAX CHEMS. MFG. CO., LTD., Kaohsiung (TW)

(21) Appl. No.: 12/856,169

(22) Filed: Aug. 13, 2010

(30) Foreign Application Priority Data
May 14, 2010 (TW) 99115408

(51) Int. Cl. A61K 8/49; A61Q 19/02; A61K 8/46; C07C 309/21; C07D 295/088

(52) U.S. Cl. 424/62; 562/106; 544/158; 562/107; 562/104

(57) ABSTRACT

The present invention provides a composition for inhibiting melanin production to promote skin whitening, which is characterized by containing compounds represented by formula (I):

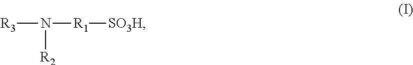

wherein R₁, R₂ and R₃ are defined as herein. The present invention also provides a method for inhibiting melanin production to promote skin whitening by using the same.

### BACKGROUND OF THE INVENTION

1. Field of the Invention

The present invention is related to a composition for inhibiting melanin production and application thereof; especially, a composition for inhibiting tyrosinase in melanocyte to inhibit melanin production and therefore promote skin whitening and application thereof.

2. Description of the Related Art

Melanin is continuously produced by melanosome of melanocyte in human body. The melanocyte is distributed in tissues such as eye, hair, brain and skin.

In skin, the melanocyte is positioned at the basal lamina of an epidermic tissue. One melanocyte is surrounded by 30˜40 keratinocyte to form so-called an epidermal melanin unit. Melanocyte has important functions in skin, although it only occupies a little parts of space.

Melanosome is continuously produced by melanocyte, and the produced melanosome will be secreted to the keratinocytes around it via the terminal of the dendritic axon of melanocyte. Melanosome is a spherical or elliptic membrane organelle in the cytoplasm of melanocyte and can protect cells from oxidative stress due to free radicals released during melanin production.

In melanosome, the production of melanin comprises a catalysis reaction of tyrosine by tyrosinase and other following enzyme-mediated catalysis reactions. Two kinds of melanin are produced; one is eumelanin with brownish black color and the other is phaeomelanin with brown color. Tyrosinase, which is a copper-contained enzyme, plays a role in the rate-determining step of melanin production.

The currently effective whitening ingredients such as vitamin C, hydroquinone, arbutin, and kojic acid, etc all have an inhibitory activity for tyrosinase. However, those ingredients may cause cell lesion and toxication during long-term usage. Thus, it is expected to develop a novel composition for inhibiting tyrosinase.

### SUMMARY OF THE INVENTION

In view of foregoing, one object of the present invention is to provide a composition for inhibiting melanin production to promote skin whitening. The composition of the present invention comprises a novel ingredient, which is a biocompatible compound with low molecular weight. Besides, the novel ingredient has inhibitory activity for tyrosinase and therefore has the ability to inhibit melanin production of B16 melanomas and human melanocytes.

Another object of the present invention is to provide a method for inhibiting melanin production to promote skin whitening.

To achieve the above objects, the present invention provides a composition for inhibiting melanin production, characterized by comprising a compound represented by formula (I):

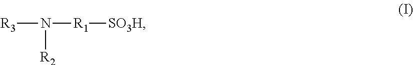

wherein R₁ is a unsubstituted or halo-substituted C2˜C10 alkylene; R₂ and R₃ are independently a hydrogen, a five- or six-membered heterocyclic or cyclic group, a C1˜C6 amidoalkyl, a C1˜C6 hydroxyalkyl or a C1˜C6 carboxyalkyl; alternatively, R₂, R₃, and the nitrogen form an oxygen-containing five- or six-membered heterocyclic group.

Preferably, said compound represented by formula (I) is a compound represented by any one of formula (I-a)˜(I-e):

(I-a): [N-(2-Acetamido)-2-aminoethanesulfonic acid, abbreviated as ACES]:

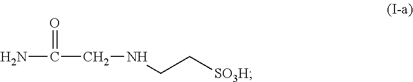

(I-b): [3-(N-Morpholino) propanesulfonic acid, abbreviated as MOPS]:

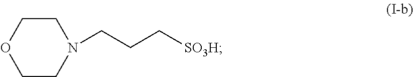

(I-c): [N,N-bis(2-hydroxyethyl)-2-aminoethanesulfonic acid, abbreviated as BES]

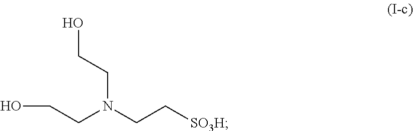

(I-d): [2-(N-Cyclohexylamino)ethanesulfonic acid, CHES]

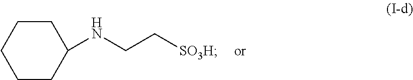

(I-e): [2-(N-Morpholino) ethanesulfonic acid, MES]

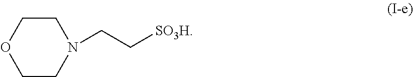

Preferably, the composition of the present invention further comprises a cosmetically acceptable excipient. Said cosmetically acceptable excipient is deionized water, a polyalcohol, a hydrophilic polymeric material or a combination thereof. The hydrophilic polymeric material comprises polyethyleneglycol (PEG), polyvinyl alcohol (PVA), starch, modified starch, xanthan gum, carrageenan, gelatin, chitosan, pectin, propolis, guar, locust bean gum, seaweed gel, collagen, cellulose derivative, modified cellulose polymer, arabinogalactan, konjac gum, fish glue, guar gum, microbial polymer, glucan, agar, alginic acid polymer or a combination thereof. The polyalcohol comprises, but not limited to propylene glycol, glycerol, sorbitol, butanediol, hexanediol, ethoxy glucoside, 1,2-hexanediol, hexanetriol or dipropylene glycol.

Preferably, a content of said compound represented by formula (I) is 0.01˜99.99 wt %.

Preferably, a content of said cosmetically acceptable excipient is 0.01˜99.99 wt %.

Preferably, said compound represented by formula (I) has an efficacy of inhibiting the activity of tyrosinase.

Preferably, the composition further comprises a commercial cosmetic or personal care product.

The present invention also provides a method for inhibiting melanin production, comprising contacting a skin with an effective amount of said composition.

Preferably, said composition is incorporated in a commercial cosmetic or personal care product before contacting with a human skin.

Yet the present invention provides a use of a compound represented by formula (I) for inhibiting melanin production:

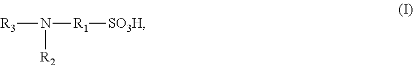

wherein R₁ is a unsubstituted or halo-substituted C2˜C10 alkylene; R₂ and R₃ are independently a hydrogen, a five- or six-membered heterocyclic or cyclic group, a C1˜C6 amidoalkyl, a C1˜C6 hydroxyalkyl or a C1˜C6 carboxyalkyl; alternatively, R₂, R₃, and the nitrogen form an oxygen-containing five- or six-membered heterocyclic group.

The composition of the present invention for inhibiting melanin production can be in a form of a skin external-use agent such as a usual cosmetic, a personal care product, a pharmaceutical product, etc; for instance, a whitening agent, a humectant, an oxidation inhibitor, an oiliness ingredient, a UV-absorbing agent, a surfactant, a tackifying agent, an alcohol, a powder ingredient, a coloring material, a hydrophilic ingredient, an aqueous solution and various skin nutrients. Other ingredients of the aforementioned forms respectively have their own formulas based on their usages. Those skilled in the art can implement the composition of the present invention for inhibiting melanin production within a proper scope.

The composition of the present invention for inhibiting melanin production achieves the efficacy of inhibiting melanin production by using a novel ingredient, which has a property of inhibiting tyrosinase activity. Incorporating the novel ingredient into a commercial personal care product or cosmetic can improve the whitening efficacy thereof.

### BRIEF DESCRIPTION OF THE DRAWINGS

FIG. 1 shows the results regarding inhibiting tyrosinase to prevent melanin production in example 1.

FIG. 2 displays the microscopic image (400×) of mouse melanoma cultured after 72 hours.

FIG. 3 illustrates the results that ACES and MOPS inhibit melanin production of B16-F1 cells.

FIG. 4 illustrates the results that ACES and MOPS inhibit melanin secretion of B16-F1 cells.

FIG. 5 shows the results that experimental samples 2-1 and 2-2 inhibit melanin production of melanocyte in human epidermal melanocyte.

FIG. 6 displays the analysis results of melanin by the digital skin analyzer (VISIA™).

FIG. 7 displays the analysis results of the amount of skin stain by the digital skin analyzer (VISIA™).

### DETAILED DESCRIPTION OF THE PRESENT INVENTION

As mentioned above, the composition of the present invention for inhibiting melanin production is characterized by comprising a compound represented by formula (I):

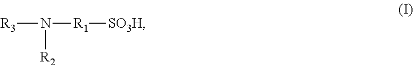

wherein R₁ is a unsubstituted or halo-substituted C2˜C10 alkylene; R₂ and R₃ are independently a hydrogen, a five- or six-membered heterocyclic or cyclic group, a C1˜C6 amidoalkyl, a C1˜C6 hydroxyalkyl or a C1˜C6 carboxyalkyl; alternatively, R₂, R₃, and the nitrogen form an oxygen-containing five- or six-membered heterocyclic group.

The method of the present invention for inhibiting melanin production specifically comprises dispersing the compound represented by formula (I) into a cosmetically acceptable excipient, and then applying it to contact with a human skin. Furthermore, the composition of the present invention is incorporated into a commercial cosmetic or personal care product before contacting with a skin.

The cosmetically acceptable excipient used in the present invention is used mainly to formulate the composition of the present invention into a proper form. Said cosmetically acceptable excipient comprises excipients which are well-known in the art, comprising, but not limited to deionized water, a polyalcohol, a hydrophilic polymeric material or a combination thereof. The hydrophilic polymeric material comprises polyethyleneglycol (PEG), polyvinyl alcohol (PVA), starch, modified starch, xanthan gum, carrageenan, gelatin, chitosan, pectin, propolis, guar, locust bean gum, seaweed gel, collagen, cellulose derivative, modified cellulose polymer, arabinogalactan, konjac gum, fish glue, guar gum, microbial polymer, glucan, agar, alginic acid polymer or a combination thereof. The polyalcohol comprises, but not limited to propylene glycol, glycerol, sorbitol, butanediol, hexanediol, ethoxy glucosside, 1,2-hexanediol, hexanetriol or dipropylene glycol.

The composition of the present invention for inhibiting melanin production can further comprise one or more of materials that are good for skins, such as a radical scavenger, an anti-oxidant, an anti-inflammatory agent, an anti-allergic agent, an anti-acne agent, a skin texture improving agent, antimicrobial agent, a whitening ingredient, and etc; and one or more auxiliary ingredients, such as an antiseptic agent, a surfactant, a thickening agent, and a beauty nutrient for changing skin color or/and improving skin condition, which is obtained by mixed with water.

The composition of the present invention inhibits melanin production by using the compound represented by formula (I) as a novel ingredient for inhibiting tyrosinase activity and therefore achieves the efficacy of whitening. Thus, it is understood that the compound represented by formula (I) can be incorporated into a commercial cosmetic or personal care product before contacting with a skin.

Accordingly, the present invention also provides a use of the compound represented by formula (I) for inhibiting melanin production.

The technical features of the present invention have already recited in the description of specification. Other materials and formulas belong to traditional knowledge in the art, and those skilled in the art can implement the present invention accordingly. The following examples are used to demonstrate the technical features and advantages of the present invention clearly.

### Example 1

### Inhibiting Tyrosinase to Prevent Melanin Production by Using the Tyrosinase Inhibitor of the Present Invention

### Methods

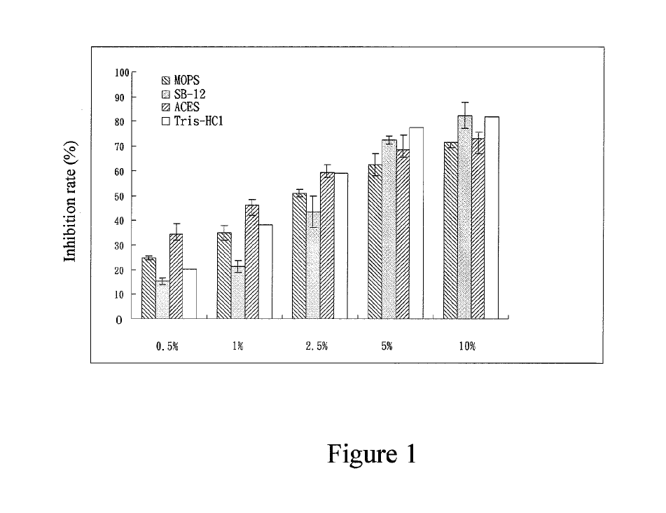

In this example, 90 μL of 0.1 U tyrosinase solution was added in to the wells of a 96-well plate. Then, 50 μL of a series diluted sample was added into the wells as an experimental group or as a blank control group, wherein said sample is MOPS, SB-12(N-Dodecyl-N,N-dimethyl-3-ammonio-1-propanesulfonate), ACES or Tris-HCl, and 0.1 M phosphate buffer and a substance lysis solution were used as blank tests for the experimental group and the blank control group, respectively. Each sample in the experiment was at least triplicated. The test plate was placed at 37° C., 400 rpm for 5 min to mix the contents thereof homogeneously. Then, the test plate was placed into a machine for an absorption examination (492 nm). An average of two detected data was obtained as a background value. The test plate was then taken out, and 60 μL of 1 mM L-Dopa solution was added into each well. After that, the test plate was placed at 37° C., 400 rpm for 15 min and then placed into the machine for another absorption examination (492 nm). At this time, an average of two detected data was obtained as a test value. The test value was minus the background value for calculating the inhibition degree, and the result was showed in FIG. 1.

### Result

In example 1, by using the tyrosinase inhibitor of the present invention, tyrosinase was inhibited to prevent melanin production. It is the main factor of skin blackening that tyrosine is eventually formed melanin after reacting with tyrosinase. During the reaction, tyrosine is transformed to L-Dopa at first, and then L-Dopa is oxidized by tyrosinase to form dopaquinone, which is then formed dopachrome by cyclization. Based on researches, dopachrome has absorption at 492 nm; thus, the oxidative activity of tyrosinase can be examined by determining the production of dopachrome. Accordingly, in this example, the absorption of the reaction at 492 nm was detected for examining the production of dopaquinone.

FIG. 1 shows the results regarding inhibiting tyrosinase to prevent melanin production in example 1. The data in the figure was calculated by the following formula:

Inhibition rate=(1−OD value of experimental group)/(OD value of blank control group)×100%

According to the results showed in the figure, the increase of the relative concentration means that the sample has the effect of the inhibiting tyrosinase activity to prevent melanin production. The results verifies that the compound of the present invention do have the effect of inhibiting tyrosinase to prevent melanin production, compared with the blank control group.

### Example 2

### Examination for Inhibiting Melanin in Cell Line Model

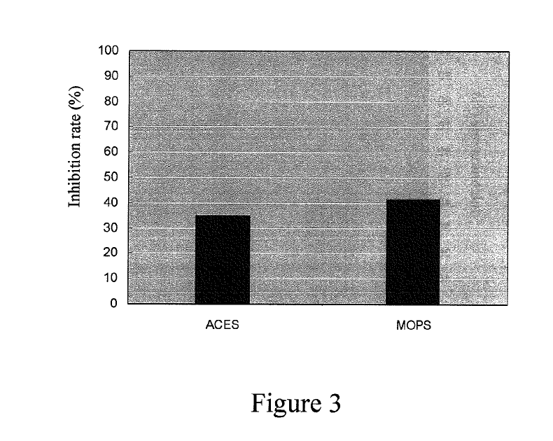

In this example, the tyrosinase inhibitor of the present invention was used to inhibit melanin production in melanocytes. The cell lines used in this example are mouse melanoma (B16-F1; ATCC CRL-6323) and human epidermal melanocyte (HEM). The culture conditions for mouse melanoma and human epidermal melanocyte in this example were listed in tables 1 and 2, respectively. Also, the additives added in every experimental sample and comparative sample were listed in table 3. The mouse melanoma and the human epidermal melanocyte were cultured in accordance with the conditions listed in tables 1˜3. After 72 hours, cells and cell culture medium were collected separately. Then, the collected cells were destroyed by ultrasonic for melanin examination. The results were showed in FIGS. 3˜5.

TABLE 1
culture conditions for mouse melanoma

| | |
|---|---|
| cell line | B16-F1 |
| medium | DMEM + 10 wt % FBS + 1 wt % P/S |
| culture condition | 37° C., 5% CO₂ |
| morphology | Epithelial-like |
| renewal of medium | Every 2~3 days |
| medium for freezing | 93 wt % medium + 7 wt % DMSO |
| sub-culture | 0.05 wt % trypsin-EDTA; 37° C., 3 minutes |

P/S represents Penicillin/Streptomycin.

TABLE 2
culture conditions for human epidermal melanocyte

| | |
|---|---|
| cell line | HEM |
| medium | Melanocyte growth medium and |
| | subculture reagent kit |
| culture condition | 37° C., 5% CO₂ |
| morphology | Fibroblast-like |
| renewal of medium | Every 2~3 days |
| medium for freezing | 93 wt % medium + 7 wt % DMSO |
| sub-culture | 0.05 wt % trypsin-EDTA; 37° C., 3 minutes |

TABLE 3
additives added in the medium

| | |
|---|---|
| Experimental | ACES (dissolved in cell culture medium, pH = 6.5-7.5, |
| sample 2-1 | 200 μg/mL) |
| Experimental | MOPS (dissolved in cell culture medium, pH = 6.5-7.5, |
| sample 2-2 | 200 μg/mL) |
| Comparative | no additives were added (blank control group) |
| sample 1 | |

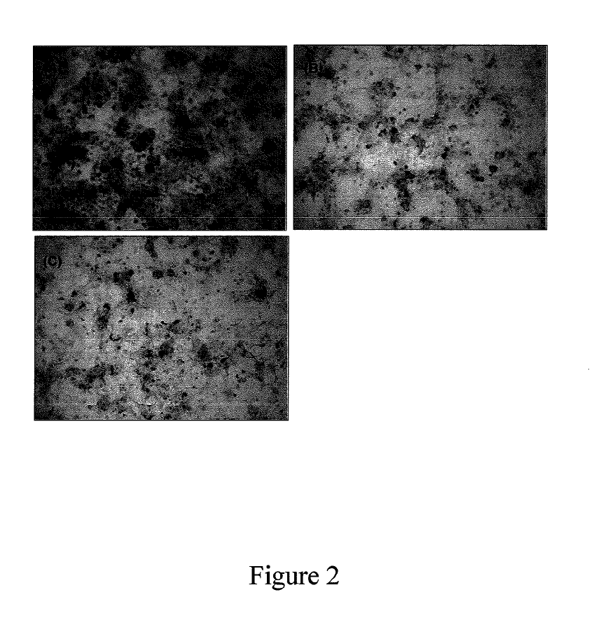

FIG. 2 displays the microscopic image (400×) of mouse melanoma cultured after 72 hours, wherein FIG. 2A is a blank control group, FIG. 2B is the group which ACES was added in the medium thereof, and FIG. 2C is the group which MOPS was added in the medium thereof. According to the results in FIG. 2, it is obvious that adding the compound of the present invention in the medium does have the efficacy of inhibiting melanin production of melanocyte. Based on the experiment of B16-F1 cells, it is found that ACES and MOPS can significantly inhibit the melanin production of mouse melanoma at a concentration of 200 μg/ml.

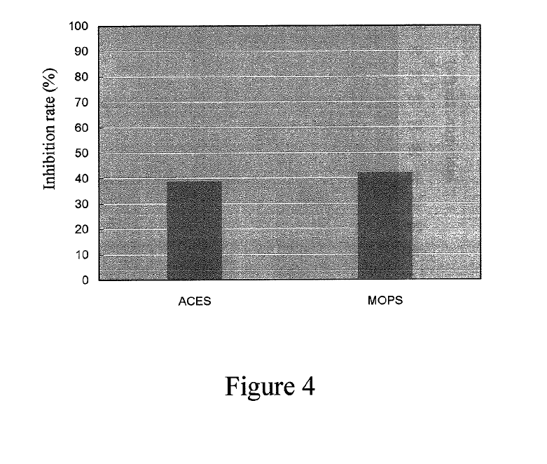

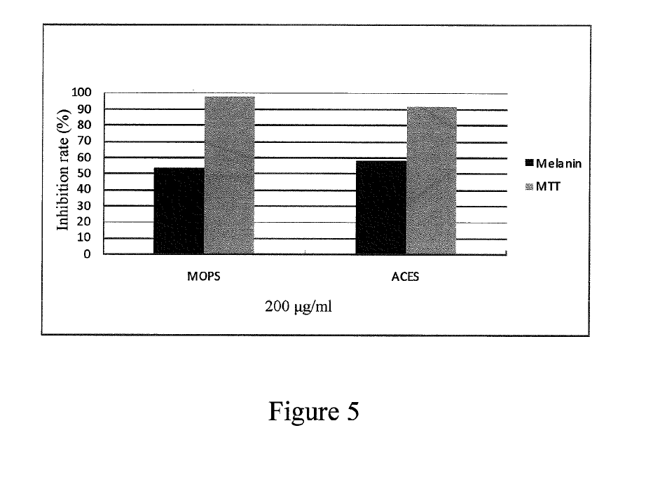

FIG. 3 illustrates the results that ACES and MOPS inhibit melanin production of B16-F1 cells. According to the results in FIG. 3, it is noted that the abilities of inhibiting melanin production of B16-F1 cells by ACES and MOPS are 35.0% and 41.7%, respectively. FIG. 4 illustrates the results that ACES and MOPS inhibit melanin secretion of B16-F1 cells. The results in FIG. 4 show that the abilities of inhibiting melanin secretion of B16-F1 cells by ACES and MOPS are 38.9% and 42.3%, respectively. FIG. 5 shows the results that experimental samples 2-1 and 2-2 inhibit melanin production of melanocyte in human epidermal melanocyte model. The increase of inhibition rate represents that the test material has the efficacy of inhibiting melanin production of human epidermal melanocyte. Based on the results showed in FIG. 5, the inhibition rate was increased by using MOPS and ACES, which means that the compound of the present invention can effectively inhibit melanin production of human epidermal melanocyte, compared with the blank control group. According to the results, it is understood that ACES and MOPS of the present invention can significantly inhibit the activity of tyrosinase, which proves that the compound of the present invention has the efficacy of inhibiting the production of melanin in melanocyte. The cell viability and activity in the above-mentioned experiments were determined by MTT assay (3-[4,5-dimethylthiahiazo-2-yl]-2,4-diphenytetrazolium bromide).

### Example 3

### Examination for Human Skin Whitening by Using the Tyrosinase Inhibitor of the Present Invention

In this example, the tyrosinase inhibitor of the present invention was used as a skin whitening agent in a form of gel for skin-maintenance and had the efficacy of skin whitening by inhibiting melanin production. The additive formulas of an experimental sample and a comparative sample of this example were listed in table 4, wherein GT-700 and Ectoin were raw materials of commercial cosmetics. GT-700 (PEG-240/HDI Copolymer Bis-Decyltetradeceth-20 Ether]; bought from KALIN ENTERPRISE CO., LTD.) is a viscosity adjustor.

TABLE 4
materials added in the formula

| | | commercial cosmetic | |
|---|---|---|---|
| | MOPS | GT-700 | Ectoin |
| Experimental sample 3 | 5 g | 0.9 g | 0.5 g |
| Comparative sample 2 | 0 g | 0.9 g | 0.5 g |

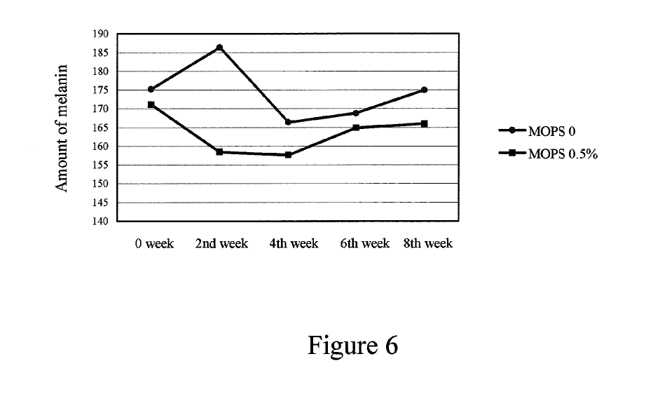

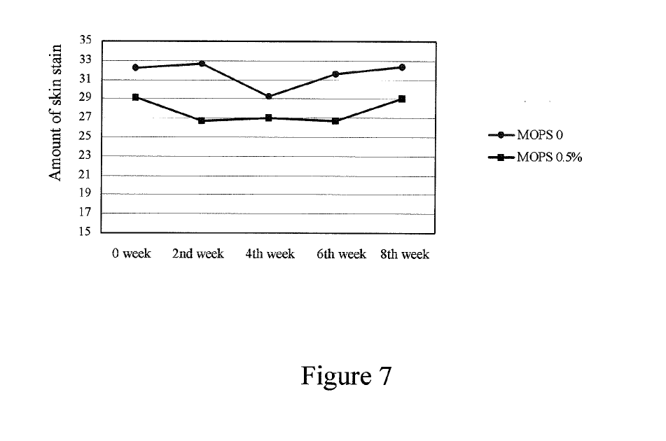

Ten testers were invited for the skin whitening experiment of this example for 8 weeks. Experimental 3 with MOPS and comparative 2 without MOPS were separately administered on left cheek and right cheek once in every morning and night. The skin color was determined by a digital skin analyzer (VISIA™) at 0, 2^{nd}, 4^{th}, 6^{th}, and 8^{th} week. A multiple spectrum image technology was involved to determine melanin, the amount of skin stain, and etc for face whitening analysis. The results were showed in FIGS. 6 and 7.

FIG. 6 displays the analysis results of melanin by the digital skin analyzer (VISIA™), and FIG. 7 displays the analysis results of the amount of skin stain by the digital skin analyzer (VISIA™). According to the results, after the 2^{nd} week, it is noted that the melanin and the amount of skin stain of experimental sample 3 were significantly less than that of comparative sample 2, and the difference was maintained to the 8^{th} week. Therefore, the results showed that the formula containing the compound represented by formula (I) of the present invention can effectively reduce melanin production; in other words, the tyrosinase inhibitor of the present invention (that is, the compound represented by formula (I)) did have the efficacy of whitening.

To sum up, the composition provided by the present invention for inhibiting melanin production comprises a novel ingredient for inhibiting tyrosinase activity and therefore has the efficacy of inhibiting melanin production. A whitening product which is different from traditional formulas can be provided by applying the novel ingredient to cosmetics or personal care products.

### Other Embodiments

The embodiments and the technical principles used are described above. All variations and modifications of the present invention and the uses thereof are included in the scope of the present invention if they do not depart from the spirit of the disclosure of this specification and drawings.

What is claimed is:

1. A composition for inhibiting melanin production, characterized by comprising a compound represented by formula (I): 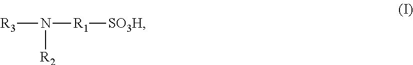
wherein R₁ is a unsubstituted or halo-substituted C2˜C10 alkylene; R₂ and R₃ are independently ahydrogen, a five- or six-membered heterocyclic or cyclic group, a C1˜C6 amidoalkyl, a C1˜C6 hydroxyalkyl or a C1˜C6 carboxyalkyl; alternatively, R₂, R₃, and the nitrogen form an oxygen-containing five- or six-membered heterocyclic group.

2. The composition according to claim 1, wherein said compound represented by formula (I) is a compound represented by any one of formula (I-a)˜(I-e):

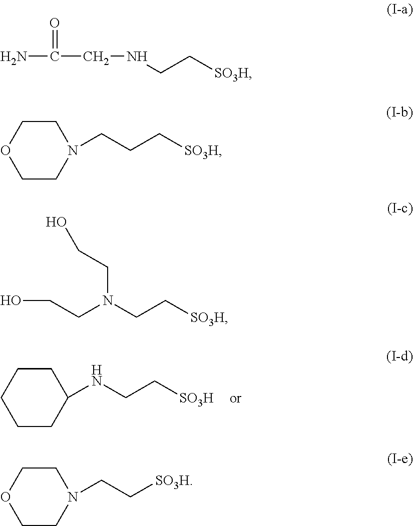

3. The composition according to claim 1, further comprising a cosmetically acceptable excipient.

4. The composition according to claim 3, wherein said cosmetically acceptable excipient is deionized water, a polyalcohol, a hydrophilic polymeric material or a combination thereof.

5. The composition according to claim 3, wherein a content of said compound represented by formula (I) is 0.01˜99.99 wt %.

6. The composition according to claim 3, wherein a content of said cosmetically acceptable excipient is 0.01˜99.99 wt %.

7. The composition according to claim 1, wherein said compound represented by formula (I) has an efficacy of inhibiting the activity of tyrosinase.

8. The composition according to claim 1, further comprising a commercial cosmetic or personal care product.

9. A method for inhibiting melanin production, comprising contacting a skin with an effective amount of said composition according to claim 1.

10. The method according to claim 9, wherein said composition is incorporated in a commercial cosmetic or personal care product before contacting with a human skin.

11. A use of a compound represented by formula (I) for inhibiting melanin production: 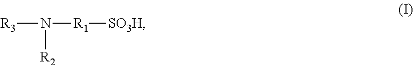
wherein R₁ is a unsubstituted or halo-substituted C2˜C10 alkylene; R₂ and R₃ are independently a hydrogen, a five- or six-membered heterocyclic or cyclic group, a C1˜C6 amidoalkyl, a C1˜C6 hydroxyalkyl or a C1˜C6 carboxyalkyl; alternatively, R₂, R₃, and the nitrogen form an oxygen-containing five- or six-membered heterocyclic group.
